(19) United States
(12) Patent Application Publication
(10) Pub. No.: US 2007/0253571 A1
(43) Pub. Date: Nov. 1, 2007

(54) SOUND REPRODUCTION DEVICE AND METHOD FOR HEARING PROTECTION IN AN AMBIENT ENVIRONMENT

(75) Inventors: Bing Li, Shenzhen (CN); Xiao-Guang Li, Shenzhen, Guangdong (CN); Wen-Sheng Tsai, Shenzhen (CN); Kuan-Hong Hsieh, Shenzhen (CN)

Correspondence Address: NORTH AMERICA INTELLECTUAL PROPERTY CORPORATION, P.O. BOX 506, MERRIFIELD, VA, 22116, US

(73) Assignee: HON HAI PRECISION INDUSTRY CO., LTD., Taipei Hsien (TW)

(21) Appl. No.: 11/619,617

(22) Filed: Jan. 4, 2007

(30) Foreign Application Priority Data
Apr. 19, 2006 (CN) 200610060399.8

(51) Int. Cl. A61F 11/06; H03G 3/20

(52) U.S. Cl. 381/72; 381/57

(57) ABSTRACT

The present invention relates to a method for hearing protection in an ambient environment of a sound reproduction device, includes steps of: receiving analog audio signals from an audio signal source via a connector; converting the analog audio signals to digital audio signals; sampling the digital audio signals to obtain a plurality of sampled amplitude values; computing an actual audio energy of the digital audio signals within a predetermined time period setting the sampled amplitude values sampled within the predetermined time period as parameters; collecting ambient noises to compute a noise level; obtaining an audio reference energy according to the noise level; comparing the actual audio energy with the audio reference energy; generating a hearing protect signal if the actual audio energy reaches the audio reference energy; and changing a current gain value or emitting reminding information, thus, protecting listeners' hearing. The present invention further provides a corresponding sound reproduction device.

### BACKGROUND OF THE INVENTION

1. Field of the Invention

The present invention relates to a sound reproduction device and method for hearing protection in an ambient environment, especially to a sound reproduction device and method for evaluating noise level, and automatically adjusting a default gain value or emitting prompt information according to the noise level.

2. Description of Related Art

The continuous development of new digital technologies has made portable audio devices (such as MP3 player) popular. When environmental noise external of the portable audio device is loud or when a favorite song is played, a listener commonly increases a gain value of the portable audio device. However listeners all have a physiological hearing threshold, i.e., loudness discomfort level (LDL). If the listener is exposed to a noise level that is larger than the listener's LDL for a long time, the listener's hearing may be impaired.

In order to solve the problems mentioned, there is a common gain control apparatus and method available in the market. The gain control apparatus provides a noise level-gain value index. The noise level-gain value index lists a plurality of gain values corresponding to noise level ranges. The gain control apparatus collects ambient noise, and computes a noise level of the ambient noise in a predetermined time field; obtaining a predetermined gain value corresponding to the noise level from the noise level-gain value index; adjusts a gain value to the predetermined gain value. Whereby the gain value of the gain control apparatus is changeable along with the noise level.

However, if a listener is in an environment where the noise level changes frequently, the gain control apparatus will frequently change the gain value accordingly. As a result, the listener will be uncomfortable. Furthermore, audio signals with different amplitudes will have different loudness at a same gain value.

Therefore, a heretofore unaddressed need exists in the industry to overcome the aforementioned deficiencies and inadequacies.

### SUMMARY OF THE INVENTION

The present invention provides a sound reproduction device and method for hearing protection while reproducing sounds in an ambient environment. The sound reproduction device and method evaluates noise level, and automatically changes a current gain value, or emitting prompt information according to the noise level.

In a first aspect of the invention, the sound reproduction device includes a connector, an audio ADC, a gain amplifier, and an electro-acoustic transducer. The connector is configured for attaching to an audio signal source. The gain amplifier receives analog audio signals from the audio signal source via the connector, and amplifying the analog audio signals. The electro-acoustic transducer reproduces prompt sounds corresponding to the amplified analog audio signals. The audio ADC receives the analog audio signals from the audio signal source via the connector, and converts the analog audio signals to digital audio signals. The sound reproduction device further includes a storage unit, a processing unit, a microphone, and a noise ADC. The microphone collects ambient noises to generate analog noise signals. The noise ADC converts the analog noise signals to digital noise signals. The storage unit stores a default gain value. The processing unit includes an amplitude sampling module, a gain obtaining module, an energy computing module, and a noise processing module. The amplitude sampling module receives the digital audio signals from the audio ADC, samples the digital audio signals to obtain a plurality of sampled amplitude values. The gain obtaining module obtains the default gain value from the storage unit. The energy computing module computes an actual audio energy of the digital audio signals within a predetermined time period according to the default gain value and the sampled amplitude values sampled within the predetermined time period. The noise processing module computes a noise level according to the digital noise signals generated by the noise ADC, obtaining an audio reference energy according to the noise level, comparing the actual audio energy with the audio reference energy, and generating a hearing protect signal if the actual audio energy reaches the audio reference energy.

In a second aspect of the invention, the sound reproduction device includes a connector, an audio ADC, and an electro-acoustic transducer. The connector is configured for attaching to an audio signal source. The electro-acoustic transducer receives analog audio signals from the audio signal source via the connector, and reproduces sounds corresponding to the amplified analog audio signals. The audio ADC receives the analog audio signals from the audio signal source via the connector, and converts the analog audio signals to digital audio signals. The sound reproduction device further includes a processing unit, a microphone, and a noise ADC. The microphone collects ambient noises to generate analog noise signals. The noise ADC converts the analog noise signals to digital noise signals. The processing unit includes an amplitude sampling module, an energy computing module and a noise processing module. The amplitude sampling module receives the digital audio signals from the audio ADC, samples the digital audio signals to obtain a plurality of sampled amplitude values. The energy computing module computes an actual audio energy of the digital audio signals within a predetermined time period according to the sampled amplitude values sampled within the predetermined time period. The noise processing module computes a noise level according to the digital noise signals generated by the noise ADC, obtaining an audio reference energy according to the noise level, comparing the actual audio energy with the audio reference energy, and generating a hearing protect signal if the actual audio energy reaches the audio reference energy.

The hearing protection method includes the steps of: receiving analog audio signals from an audio signal source via a connector; converting the analog audio signals to digital audio signals; sampling the digital audio signals to obtain a plurality of sampled amplitude values of the digital audio signals; computing an actual audio energy of the digital audio signals within a predetermined time period setting the sampled amplitude values sampled within the predetermined time period as parameters; collecting ambient noises to compute a noise level; obtaining an audio reference energy according to the noise level; comparing the actual audio energy with the audio reference energy; and generating a hearing protect signal if the actual audio energy reaches the audio reference energy.

Other systems, methods, features, and advantages will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a schematic diagram of a hardware infrastructure of a sound reproduction device for hearing protection in an ambient environment in accordance with a first preferred embodiment of the present invention;

FIG. 2 is a block diagram of a hardware infrastructure of the sound reproduction device of FIG. 1 in accordance with the first preferred embodiment of the present invention;

FIG. 3 is a schematic diagram of main function modules of a processing unit of FIG. 2;

FIG. 4 is a flowchart of a preferred hearing protection method in the ambient environment by utilizing the sound reproduction device of FIG. 2;

FIG. 5 is a schematic diagram of a hardware infrastructure of a sound reproduction device for hearing protection in the ambient environment in accordance with a second preferred embodiment of the present invention;

FIG. 6 is a block diagram of a hardware infrastructure of the sound reproduction device of FIG. 5 in accordance with the second preferred embodiment of the present invention;

FIG. 7 is a schematic diagram of main function modules of a processing unit of FIG. 6; and

FIG. 8 is a block diagram of a hardware infrastructure of the sound reproduction device in accordance with a third preferred embodiment of the present invention.

### DETAILED DESCRIPTION OF THE INVENTION

In the following embodiments, for simplicity, a hearing protection function incorporated in a sound reproduction device, such as an earphone, is depicted. The sound reproduction device of the present invention allows hearing protection while reproducing sounds in an ambient environment. The following detailed description of the embodiments is made with reference to the attached drawings.

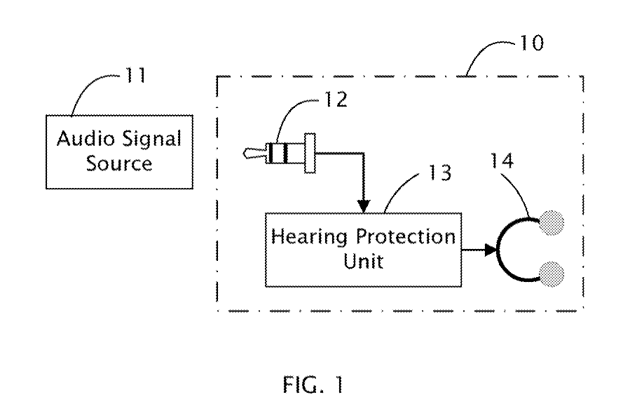

FIG. 1 is a schematic diagram of a hardware infrastructure of the sound reproduction device for hearing protection in accordance with a first preferred embodiment of the present invention. The sound reproduction device 10 includes a connector 12, a hearing protection unit 13, and an electro-acoustic transducer 14. The audio signal source 11 may be a music player, a radio player, a TV set, and so on. The sound reproduction device 10 receives analog audio signals from an audio signal source 11 via the connector 12, and sends the analog audio signals to the hearing protection unit 13.

The hearing protection unit 13 is configured for converting the analog audio signals to digital audio signals, computes an audio energy of the digital audio signals within a predetermined time period, and when the audio energy reaches a predetermined value, automatically changes a default gain value to a reduced gain value.

The electro-acoustic transducer 14 receives the analog audio signal sent from the audio signal source 11, and reproduces sounds corresponding to the analog audio signals. The electro-acoustic transducer 14 may be an earphone or a speaker.

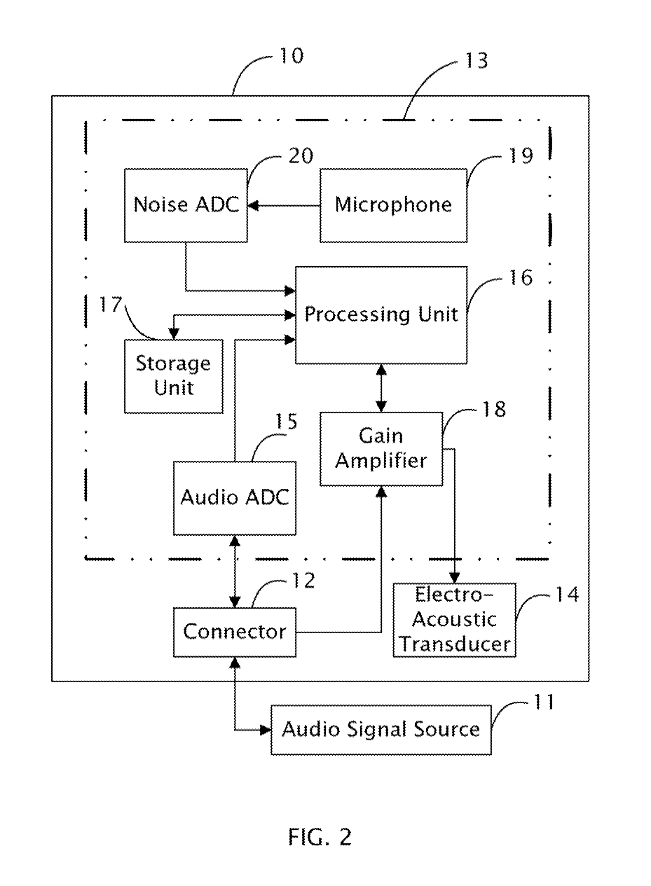

FIG. 2 is a block diagram of a hardware infrastructure of the sound reproduction device of FIG. 1 in accordance with the second preferred embodiment. The hearing protection unit 13 includes an audio analog-to-digital converter (ADC) 15, a processing unit 16, a storage unit 17, and a gain amplifier 18. The storage unit 17 stores a default gain value. The storage unit 17 may be a flash storage, a hard disk driver, and the like.

The gain amplifier 18 is configured for receiving and amplifying the analog audio signals from the audio signal source 11 via the connector 12, thereby yielding amplified analog audio signals that is sent to the electro-acoustic transducer 14. The audio ADC 15 receives the analog audio signals from the audio signal source 11 via the connector 12, converts the analog audio signals to digital audio signals, and sends the digital audio signals to the processing unit 16.

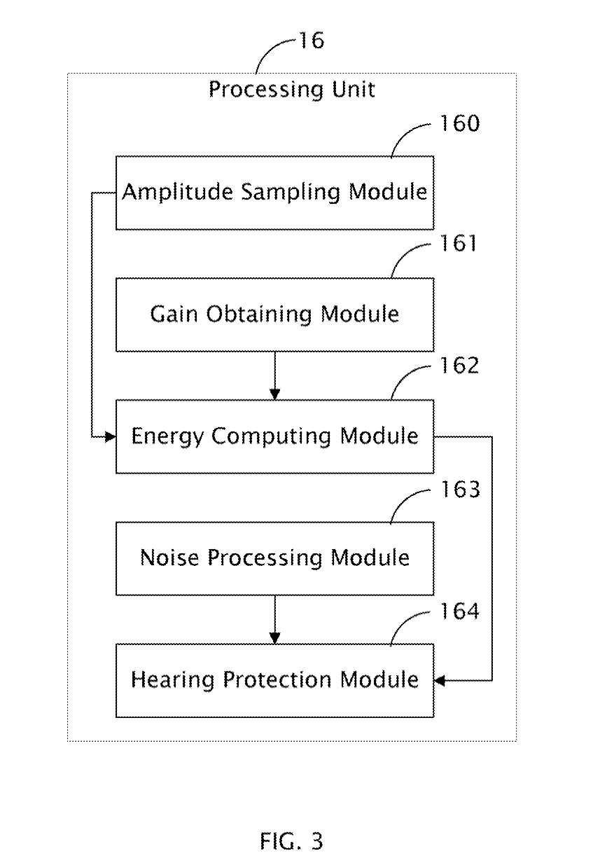

Referring to FIG. 3, the processing unit 16 includes an amplitude sampling module 160, a gain obtaining module 161, an energy computing module 162, a noise processing module 163, and a hearing protection module 164.

The amplitude sampling module 160 receives the digital audio signals from the audio ADC 15, and samples the digital audio signals to obtain a plurality of sampled amplitude values of the digital audio signals. The gain obtaining module 161 obtains the default gain value from the storage unit 17.

The energy computing module 162 computes an actual audio energy of the digital audio signals within the predetermined time period by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

The hearing protection unit 13 further includes a microphone 19 and a noise ADC 20. The microphone 19 is configured for collecting ambient noises, and for generating analog noise signals. The noise ADC 20 is configured for converting the analog noise signals to digital noise signals.

The noise processing module 163 computes a noise level according to the digital noise signals, and computes an audio reference energy according to the noise level. In a first preferred method, the noise processing module 163 subtracts a predetermined noise level from the noise level to obtain a margin, divides the margin by the predetermined noise level to obtain the changed ratio, i.e., changed ratio=(noise level−predetermined noise level)/predetermined noise level, and multiplies the changed ratio with a predetermined audio energy to obtain the audio reference energy.

In an alternative preferred method, the storage unit 17 further stores a noise level-audio energy index. The noise level-audio energy index is a table that lists the audio reference energys according to noise level ranges. The noise processing module 163 searches the noise level-audio energy index with the noise level to obtain the audio reference energy correspondingly.

The noise processing module 163 compares the actual audio energy with the audio reference energy, and generates a hearing protect signal if the actual audio energy reaches the audio reference energy. The reference audio energy is an upper threshold value (loudest sound intensity) appropriate for a listener.

When the hearing protection module 164 receives the hearing protect signal, the hearing protection module 164 automatically changes the default gain value to the reduced gain value, signals the gain amplifier 18 to amplify the analog audio signals received from the audio signal source 11 with the reduced gain value, and updates the default gain value in the storage unit 17 with the reduced gain value.

In an alternative preferred embodiment, when the hearing protection module 164 receives the hearing protect signal the hearing protection module 164 sends prompt signals to the gain amplifier 18. The gain amplifier 18 is configured for receiving and amplifying the prompt signals with the default gain value, thereby yielding amplified prompt signals that is then sent to the electro-acoustic transducer 14. The electro-acoustic transducer 14 receives the amplified prompt signals, and reproduces prompt sounds according to the amplified prompt signals. The prompt sounds are used for alerting the listener to manually reduce a current gain value of the audio signal source 11.

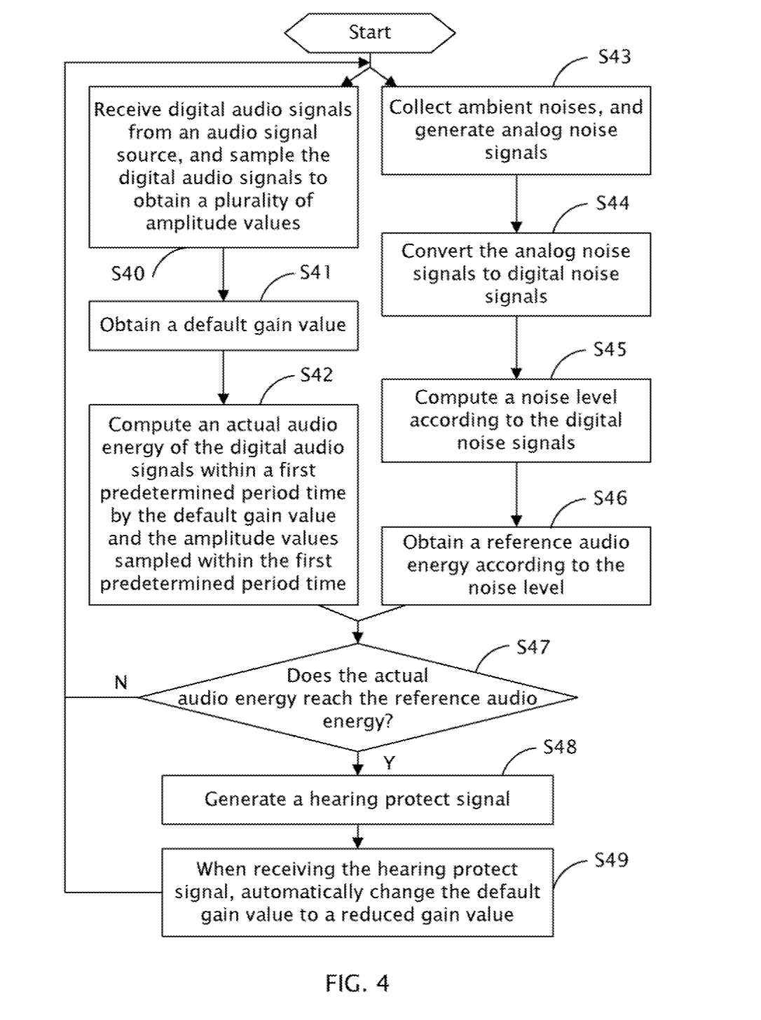

FIG. 4 is a flowchart of a first preferred method for hearing protection in the ambient environment by utilizing the sound reproduction device of FIG. 2. In step S40, the amplitude sampling module 160 receives the digital audio signals from the audio ADC 15, and samples the digital audio signals to obtain the plurality of sampled amplitude values of the digital audio signals.

In step S41, the gain obtaining module 161 obtains the default gain value from the storage unit 17.

In step S42, the energy computing module 162 computes the actual audio energy of the digital audio signals within the predetermined time period according to the default gain value and the sampled amplitude values sampled within the predetermined time period.

Simultaneously with step S42, in step S43, the microphone 19 collects the ambient noises from the ambient environment, and generate the analog noise signals.

In step S44, the noise ADC 20 converts the analog noise signals to the digital ambient noises.

In step S45, the noise processing module 163 computes the noise level according to the digital noise signals converted by the noise ADC 20.

In step S46, the noise processing module 163 reads the audio reference energy corresponding to the noise level from the noise level-audio energy index, or alternatively computes the audio reference energy.

In step S47, the noise processing module 163 compares the actual audio energy with the audio reference energy, detects whether the actual audio energy reaches the audio reference energy. If the actual audio energy does not reach the audio reference energy, the procedure turns to start.

When the actual audio energy reaches the audio reference energy, in step S48, the noise processing module 163 generates the hearing protect signal.

In step S49, when the hearing protection module 164 receives the hearing protect signal, the hearing protection module 164 automatically changes the default gain value to the reduced gain value, signals the gain amplifier 18 to amplify the analog audio signals received from the audio signal source 11 by the reduced gain value, and updates the default gain value stored in the storage unit 17 with the reduced gain value, then the procedure turns to start.

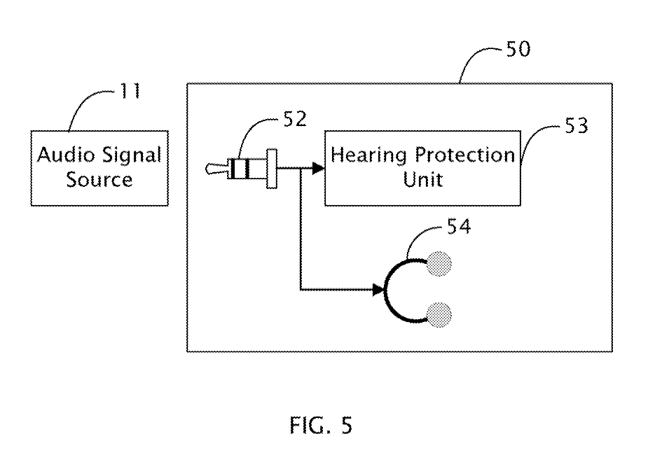

FIG. 5 is a schematic diagram of a hardware infrastructure of the sound reproduction device for hearing protection in accordance with a second preferred embodiment of the present invention. The sound reproduction device 50 includes a connector 52, a hearing protection unit 53, and an electro-acoustic transducer 54. The sound reproduction device 50 receives the analog audio signals from the audio signal source 11 via the connector 52, and sends the analog audio signals to the hearing protection unit 53 and the electro-acoustic transducer 54 respectively.

The hearing protection unit 53 converts the analog audio signals to digital audio signals, computes the audio energy of the digital audio signals within the predetermined time period, and when the audio energy reaches the predetermined value, reproduces the prompt indicator to alert the listener to manually reduce the current gain value of the audio signal source, thus, preventing hearing impairment of the listener. The prompt indicator is selected from the group consisting of visual indicator and acoustical indicator.

The electro-acoustic transducer 54 reproduces sounds corresponding to the analog audio signals. The electro-acoustic transducer 54 may be an earphone or a speaker.

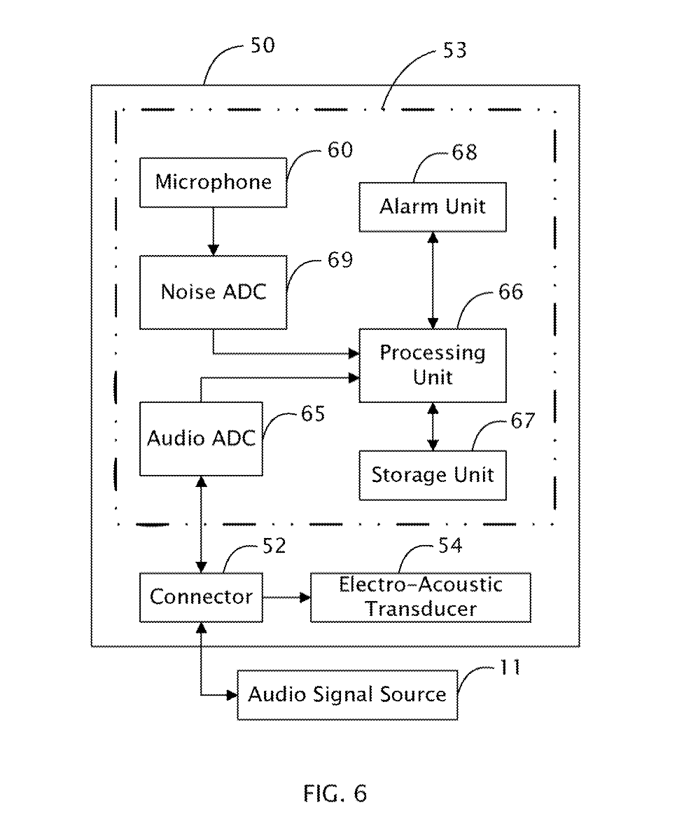

FIG. 6 is a block diagram of a hardware infrastructure of the sound reproduction device 50 of FIG. 5 in accordance with the second preferred embodiment. The hearing protection unit 53 includes an audio ADC 65, a processing unit 66, a storage unit 67 and an alarm unit 68. The alarm unit 68 may be an acoustical indicating device such as a buzzer, or a visual indicating device such as an LED (light-emitting diode).

The audio ADC 65 receives the analog audio signals from the audio signal source 11 via the connector 52, converts the analog audio signals to the digital audio signals, and sends the digital audio signals to the processing unit 66.

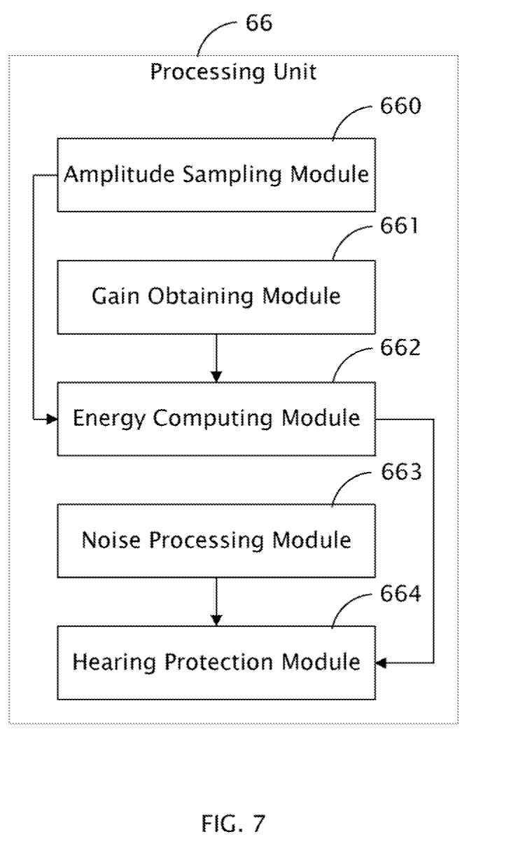

Referring to FIG. 7, the processing unit 66 includes an amplitude sampling module 660, a gain obtaining module 661, an energy computing module 662, a noise processing module 663, and a hearing protection module 664.

The amplitude sampling module 660 receives the digital audio signals from the audio ADC 65, samples the digital audio signals at a predetermined frequency, and obtains a plurality of sampled amplitude values of the digital audio signals.

The energy computing module 661 periodically computes an audio energy within the predetermined time period by applying a formula as follows: Q=[Σ(mi)²/N]^{1/2}*T, wherein Q represents the actual audio energy, mi presents the sampled amplitude values sampled within the predetermined time period, N represents a count of the sampled amplitude values sampled within the predetermined time period, and i denotes an identifier of the sampled amplitude value.

The hearing protection unit 63 further includes a microphone 60 and a noise ADC 69. The microphone 60 is configured for collecting ambient noises, and generating analog noise signals. The noise ADC 60 is configured for converting the analog noise signals to digital noise signals.

The noise processing module 663 computes the noise level according to the digital noise signals, and obtains the audio reference energy corresponding to the noise level. In a preferred method, the noise processing module 663 subtracts the predetermined noise level from the noise level to obtain the margin, divides the margin by the predetermined noise level to obtain the changed ratio i.e., changed ratio=(noise level−predetermined noise level)/predetermined noise level, and multiplies the changed ratio with the predetermined audio energy to obtain the audio reference energy.

In an alternative preferred method, the hearing protection unit 63 further includes a storage unit 67 for storing the noise level-audio energy index. The noise level-audio energy index is the table that lists the audio reference energy corresponding to noise level ranges. The noise processing module 663 reads the noise level-audio energy index to obtain the audio reference energy corresponding to the noise level.

The noise processing module 663 compares the actual audio energy with the audio reference energy, and generates a hearing protect signal when the actual audio energy reaches the audio reference energy.

The hearing protection module 664, when receiving the hearing protect signal, signals the alarm unit 68 to output prompt information. The prompt information is used for alerting the listener to manually reduce the current gain value of the audio signal source 11, thus, preventing hearing impairment of the listener.

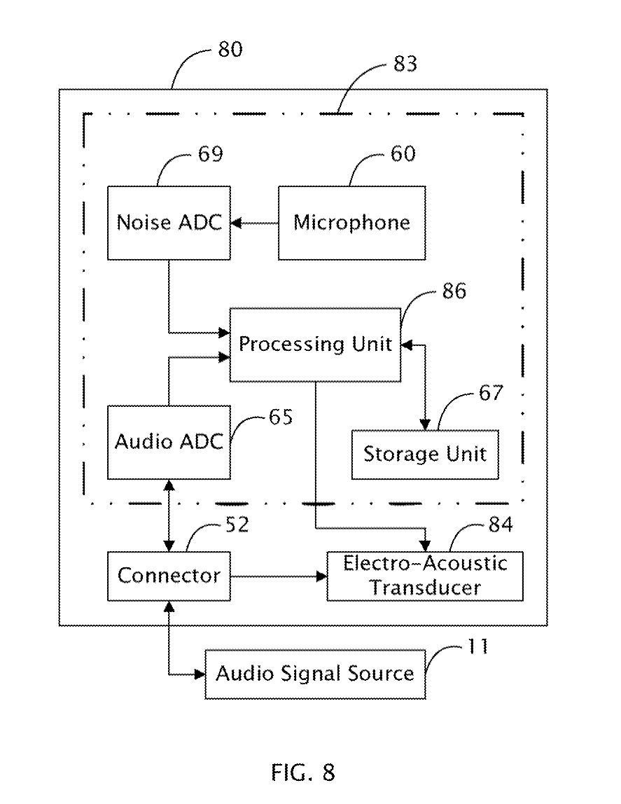

In a third preferred embodiment, referring to FIG. 8, the difference between the third embodiment and the second embodiment is that the hearing protection unit 83 of the third embodiment does not include the alarm unit 68, and a processing unit 86 and an electro-acoustic transducer 84 is adopted to perform the same functions of the alarm unit 68.

The processing unit 86 includes an amplitude sampling module 860, a gain obtaining module 861, an energy computing module 862, and a noise processing module 863, which respectively performs the same functions as the amplitude sampling module 160, the gain obtaining module 161, the energy computing module 162, and the noise processing module 163 of the processing unit 16 in the first and second embodiment.

The processing unit 86 further includes a hearing protection module 864. The hearing protection module 864, when receiving the hearing protect signal, sends the prompt signals to the electro-acoustic transducer 84. The electro-acoustic transducer 84 reproduces prompt sounds corresponding to the prompt signals.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. A sound reproduction device for hearing protection in an ambient environment, the sound reproduction device comprising:
a connector configured for attaching to an audio signal source;
a gain amplifier configured for receiving and amplifying analog audio signals from the audio signal source via the connector, thereby yielding amplified analog audio signals;
an electro-acoustic transducer configured for reproducing sounds corresponding to the amplified analog audio signals;
an audio ADC configured for receiving the analog audio signals from the audio signal source via the connector, and converting the analog audio signals to digital audio signals;
a microphone configured for collecting ambient noises, and generating analog noise signals;
a noise ADC configured for converting the analog noise signals to digital noise signals;
a storage unit configured for storing a default gain value; and
a processing unit comprising:
an amplitude sampling module for receiving the digital audio signals from the audio ADC, and sampling the digital audio signals to obtain a plurality of sampled amplitude values;
a gain obtaining module for obtaining the default gain value from the storage unit;
an energy computing module for computing an actual audio energy of the digital audio signals within a predetermined time period according to the default gain value and the sampled amplitude values sampled within the predetermined time period; and
a noise processing module for computing a noise level according to the digital noise signals generated by the noise ADC, obtaining an audio reference energy according to the noise level, comparing the actual audio energy with the audio reference energy, and generating a hearing protect signal if the actual audio energy reaches the audio reference energy.

2. The sound reproduction device according to claim 1, wherein the processing unit further comprises a hearing protection module, when receiving the hearing protect signal, the hearing protection module automatically changes the default gain value to a reduced gain value, signals the gain amplifier to amplify the received analog audio signals by the reduced gain value, and updates the default gain value stored in the storage unit with the reduced gain value.

3. The sound reproduction device according to claim 1, wherein the processing unit further comprises a hearing protection module, when receiving the hearing protect signal, the hearing protection module sends prompt signals to the gain amplifier, the gain amplifier amplifies the prompt signals thereby yielding amplified prompt signals, and the electro-acoustic transducer reproduces prompt sounds corresponding to the amplified prompt signals.

4. The sound reproduction device according to claim 1, wherein:
the storage unit further stores a noise level-audio energy index, the noise level-audio energy index listing a plurality of audio reference energy corresponding to noise level ranges; and
the noise processing module reads the noise level-audio energy index to obtain the audio reference energy corresponding to the noise level.

5. The sound reproduction device according to claim 1, wherein the noise processing module subtracts a predetermined noise level from the noise level to obtain a margin, divides the margin by the predetermined noise level to get a changed ratio, and multiplies a predetermined audio energy by the changed ratio to obtain the audio reference energy.

6. The sound reproduction device according to claim 1, wherein the actual audio energy is computed by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

7. A sound reproduction device for hearing protection in an ambient environment, the sound reproduction device comprising:
a connector configured for attaching to an audio signal source;
an electro-acoustic transducer configured for receiving analog audio signals from the audio signal source via the connector, and reproducing sounds corresponding to the analog audio signals;
an audio ADC configured for receiving the analog audio signals from the audio signal source via the connector, and converting the analog audio signals to digital audio signals;
a microphone configured for collecting ambient noises, and generating analog noise signals;
a noise ADC configured for converting the analog noise signals to digital noise signals; and
a processing unit comprising:
an amplitude sampling module for receiving the digital audio signals from the audio ADC, sampling the digital audio signals to obtain a plurality of sampled amplitude values;
an energy computing module for computing an actual audio energy of the digital audio signals within a predetermined time period according to the sampled amplitude values sampled within the predetermined time period; and
a noise processing module for computing a noise level according to the digital noise signals generated by the noise ADC, obtaining an audio reference energy according to the noise level, comparing the actual audio energy with the audio reference energy, generating a hearing protect signal if the actual audio energy reaches the audio reference energy.

8. The sound reproduction device according to claim 7, wherein the processing unit further comprises a hearing protection module, when receiving the hearing protect signal, the hearing protection module sends prompt signals to the electro-acoustic transducer, and the electro-acoustic transducer reproduces prompt sounds corresponding to the prompt signals.

9. The sound reproduction device according to claim 7, further comprising an alarm unit, wherein the processing unit further comprises a hearing protection module, when receiving the hearing protect signal, the hearing protection module signals the alarm unit to output prompt information.

10. The sound reproduction device according to claim 9, wherein the prompt information is selected from the group consisting of visual reminding information and acoustical reminding information.

11. The sound reproduction device according to claim 7, further comprising a storage unit, wherein the storage unit stores a noise level-audio energy index for listing audio reference energy corresponding to noise level ranges; and the noise processing module reads the noise level-audio energy index to obtain the audio reference energy corresponding to the noise level.

12. The sound reproduction device according to claim 7, wherein the noise processing module subtracts a predetermined noise level from the noise level to obtain a margin, divides the margin by the predetermined noise level to get a changed ratio, and multiplies a predetermined audio energy by the changed ratio to obtain the audio reference energy.

13. The sound reproduction device according to claim 7, wherein the actual audio energy is computed by: Q=[Σ(mi)²/N]^{1/2}, wherein Q represents the actual audio energy, mi represents the sampled amplitude values sampled within the predetermined time period, N represents a count of the sampled amplitude values sampled within the predetermined time period, and i denotes an identifier of the sampled amplitude value.

14. A hearing protection method in an ambient environment of a sound reproduction device, the method comprising the steps of:
receiving analog audio signals from an audio signal source via a connector;
converting the analog audio signals to digital audio signals;
sampling the digital audio signals to obtain a plurality of sampled amplitude values of the digital audio signals;
computing an actual audio energy of the digital audio signals within a predetermined time period setting the sampled amplitude values sampled within the predetermined time period as parameters;
collecting ambient noises to compute a noise level;
obtaining an audio reference energy corresponding to the noise level;
comparing the actual audio energy with the audio reference energy; and
generating a hearing protect signal if the actual audio energy reaches the audio reference energy.

15. The hearing protection method according to claim 14, wherein the actual audio energy is computed by: Q=[Σ(mi)²/N]^{1/2}, wherein Q represents the actual audio energy, mi represents the sampled amplitude values sampled within the predetermined time period, N represents a count of the sampled amplitude values sampled within the predetermined time period, and i denotes an identifier of the sampled amplitude value.

16. The hearing protection method according to claim 14, further comprising the step of: obtaining a default gain value acting as a parameter for computing the actual audio energy by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

17. The hearing protection method according to claim 16, further comprising the steps of: when receiving the hearing protect signal, automatically changing the default gain value to a reduced gain value, amplifying the received analog audio signals by the reduced gain value, and updating the default gain value with the reduced gain value.

18. The hearing protection method according to claim 14, wherein the step of obtaining the audio reference energy comprises the steps of:
providing a storage unit for storing a noise level-audio energy index, the noise level-audio energy index storing audio reference energy corresponding to noise level ranges; and
reads the noise level-audio energy index to obtain the audio reference energy corresponding to the noise level.

19. The hearing protection method according to claim 14, wherein the step of obtaining the audio reference energy comprises the steps of:
subtracting a predetermined noise level from the noise level to obtain a margin;
dividing the margin by the predetermined noise level to get a changed ratio; and
multiplying a predetermined audio energy by the changed ratio to obtain the audio reference energy.

20. The hearing protection method according to claim 14, further comprising the steps of: when receiving the hearing protect signal, emitting prompt information.

21. The hearing protection method according to claim 20, wherein the prompt information is selected from the group consisting of visual reminding information and acoustical reminding information.
